(19) United States
(12) Patent Application Publication
(10) Pub. No.: US 2012/0308619 A1
(43) Pub. Date: Dec. 6, 2012

(54) COSMETIC ANTI-WRINKLE PATCH

(76) Inventor: Catherine L.B. Tousley, Seattle, WA (US)

(21) Appl. No.: 13/486,252

(22) Filed: Jun. 1, 2012

Related U.S. Application Data
(60) Provisional application No. 61/493,253, filed on Jun. 3, 2011.

(51) Int. Cl. A61K 8/02; A61K 8/81; A61Q 19/08; A61K 8/891

(52) U.S. Cl. 424/401

(57) ABSTRACT

A cosmetic patch is made of a thin adhesive-backed translucent polymer film tape releasably adhered to a release liner sheet, the polymer film tape being die-cut to provide at least one adhesive-backed removable section thereof sized shaped to fit a skin region of a wearer's face to inhibit creasing of the skin during sleep.

### RELATED APPLICATION

This application claims the benefit under 35 U.S.C. § 119(e) from U.S. Provisional Application No. 61/493,253, filed Jun. 3, 2011, which is incorporated herein by reference.

### TECHNICAL FIELD

The field of the present disclosure relates to cosmetic aids in general, and more specifically to an anti-wrinkle patch.

### BACKGROUND

Not only does the natural process of aging cause wrinkles, so does sun damage, free radicals, hormones, muscle use, and gravity. One area that noticeable wrinkling occurs is the glabellar lines, which are the vertical lines between the eyebrows. These lines appear when a person frowns due to excessive sun exposure and/or habitual frowning. This area can also be affected by sleep habits. Sleeping on one's side or stomach causes compression of the face and creasing of these muscles. Good nutrition and exercise, and by reducing exposure to sun can help slow the process of wrinkling, as can a variety of treatments. Depending upon the degree of the condition and a patient's motivation, there is an array of options, ranging from noninvasive to invasive surgery.

One method of reducing the effect of frown lines is DermaGen Anti-Wrinkle Patches. According to the manufacturer, the DermaGen patches are designed to minimize and flatten raised scar areas, while also reducing wrinkles.

Another method is Frownies, facial pads that require they be wetted prior to application and for removal. This is a wrinkle treatment the manufacturer says, addresses the physical cause of deep lines and wrinkles in the skin by training the underlying facial muscles to regain the strength and tone they have lost.

Anti Aging Wrinkle Creams may also reduce the appearance of fine lines and wrinkles with active ingredients such as antioxidants and vitamins.

An invasive option is Botox, which is a protein derived from botulism toxin that is injected underneath skin in order to minimize or smooth out lines and wrinkles on the face. It works on a temporary basis, and results usually last from 3-6 months and is expensive.

The most drastic alternative is a face-lift, a plastic surgery procedure that has the ability to remove excess fat, tighten muscles, and reshape the skin of the face, resulting in a more youthful appearance.

### SUMMARY

An anti-wrinkle patch according to the present disclosure offers a novel method of reducing the appearance of glabellar lines, at least temporarily. The patches are placed between the eyes to hold skin smoothly in place during waking hours and/or to keep skin from compressing while asleep. Reduction in the appearance of these lines may be seen in as little as a few hours but is more significant with overnight use.

The patch is noninvasive, hypoallergenic, and latex-free with no added chemicals. It can be used in conjunction with most skin creams, or can be used solely on its own. By not introducing added chemicals to the skin, the patch allows for a chemical-free alternative to other products and invasive methods such as Botox.

The patch may be made of a translucent polymer film so that it is almost invisible when worn on the skin. Other known patches are made out of paper or opaque materials, making them more obvious when worn and aesthetically undesirable.

Additional aspects and advantages will be apparent from the following detailed description of preferred embodiments, which proceeds with reference to the accompanying drawings.

### BRIEF DESCRIPTION OF THE DRAWINGS

FIG. 1 is a front view of a cosmetic facial patch according to an embodiment;

FIG. 2 is a front view of the cosmetic facial patch of FIG. 1, shown applied to a user's forehead to relax wrinkles and fines lines;

FIG. 3 is a front view of a sheet of three of the cosmetic facial patches of FIG. 1; and

FIG. 4 is a side view of the sheet of FIG. 3 showing one of the patches being peeled away from a release liner.

### DETAILED DESCRIPTION OF PREFERRED EMBODIMENTS

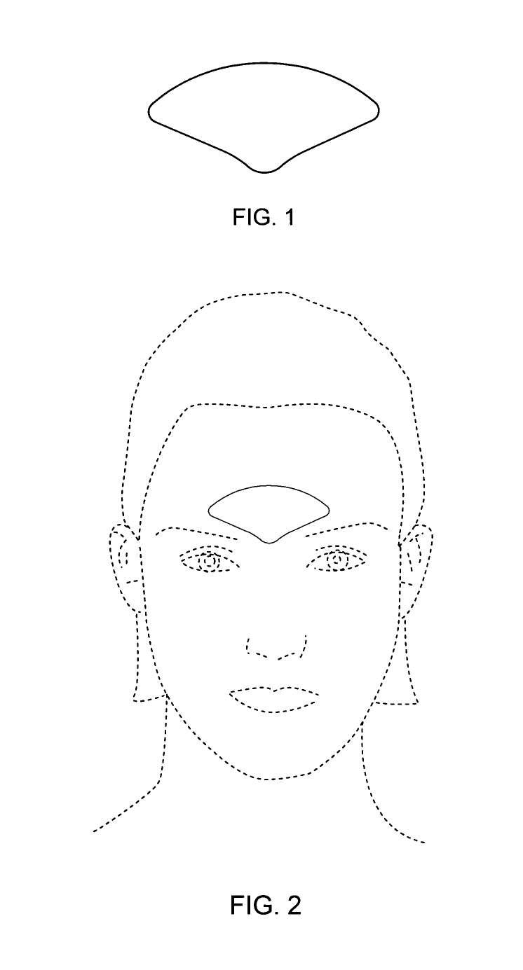

FIG. 1 depicts a cosmetic facial patch 10 in accordance with a preferred embodiment. Patch 10 is configured in a wedge or “fan” shape, with an arcuate upper edge 12 and left and right inclined edges 14, 16 which converge to a lowermost central root portion 18, which is radiused. Root portion 18 has a protruding bulbous shape that extends beyond the normal intersection of lines coincident with edges 14, 16, which provides improved patch coverage between the eyebrows and may also ease removal of the patch 10, as described below.

Referring to FIG. 2 we see a front view of a person 30 wearing the patch 10. The patch 10 is placed between the person's eyebrows to hold skin in the glabella region 20 smoothly in place during waking hours and/or to keep skin from compressing while sleeping. The shape of the patch 10 is designed to not only fit comfortably on the forehead area and in the glabella region between the eyebrows, but to also taper at the edges 12, 14 to minimize creasing and pressure on the skin, as not to create additional wrinkles.

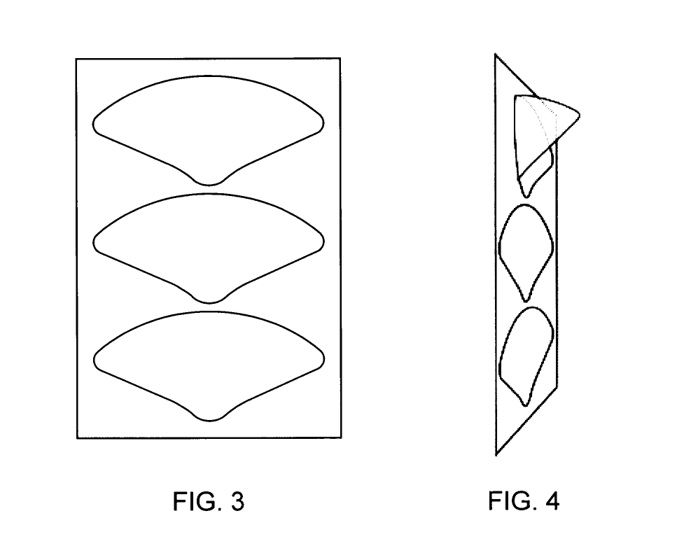

Patch 10 is preferably formed of a thin adhesive-backed translucent polyer film tape that is hypoallergenic, latex-free, and suitable for daily usage. The polymer film tape is preferably a polyethylene film having a thickness of between 6 and 7 mils and more preferably about 6.4 mils (0.16 mm). The film may be transparent or, more preferably, translucent with a matte finish to reduce reflections, making it almost invisible when worn. As illustrated in FIG. 3, patches 10 may be delivered as a sheet 40 of three patches 10 die-cut from and releasably adhered to a release liner 42, which forms a backing sheet or substrate for the patches 10. Relase liner 42 may be poly-coated craft paper with a silicone layer providing release capability for the adhesive backing of the patch 10. FIG. 4 is a side perspective view of the sheet 40 illustrating one of the patches 10 being peeled away from the release liner 42.

The adhesive of the self-adhering patch 10 has an adhesion value that allows for general ease of application and removal from skin. A preferred self-adhesive patch 10 has an operable adhesion to steel (peel force) of between 18 to 24 ounces/inch width, and more preferably about 21 ounces/inch width, so that the patch 10 has a comfortable medium adhesion to facial skin. One suitable adhesive is acrylate of the kind designed for medical and surgical uses, although other adhesives may also be used. The adhesive may have an applied thickness of 1 to 2 mils and more preferably about 1.4 mils. Patch 10 can remain on skin for as little or long as desired up to 24 hours at a time, and can be easily removed from skin by pulling the patch across itself 180° and peeling it off.

It will be obvious to those having skill in the art that many changes may be made to the details of the above-described embodiments without departing from the underlying principles of the invention. The scope of the present invention should, therefore, be determined only by the claims.

1. A cosmetic patch, comprising:
a release liner sheet; and
a thin translucent polymer film tape having a thickness between 6 and 7 mils and an adhesive backing layer releasably adhering the film tape to the release liner sheet, the polymer film tape being die-cut to provide at least one adhesive-backed removable section thereof sized and shaped to fit a skin region of a wearer's face to inhibit creasing of the skin during sleep.

2. The cosmetic patch of claim 1, wherein the adhesive-backed removable section is sized and shaped to fit the wearer's forehead and to inhibit skin in the wearer's glabella region from creasing during sleep.

3. The cosmetic patch of claim 1, wherein the film tape is hypoallergenic and latex-free.

4. The cosmetic patch of claim 1, wherein the film tape is transparent.

5. The cosmetic patch of claim 1, wherein the film tape has a matte finish on an outer side opposite its adhesive backing.

6. The cosmetic patch of claim 1, wherein the release liner sheet includes a silicone layer facing the film tape.

7. The cosmetic patch of claim 1, wherein the adhesive backing of the film tape comprises an acrylate adhesive having an applied thickness of 1 to 2 mils.
